(19) United States
(12) Patent Application Publication
(10) Pub. No.: US 2026/0069844 A1
(43) Pub. Date: Mar. 12, 2026

(54) Microneedle Application Tools and Methods

(71) Applicant: Georgia Tech Research Corporation, Atlanta, GA (US)

(72) Inventors: Mark R. Prausnitz, Atlanta, GA (US); Erkan Azizoglu, Atlanta, GA (US)

(21) Appl. No.: 19/107,375

(22) PCT Filed: Sep. 13, 2023
(86) PCT No.: PCT/US2023/032663

§ 371 Date: Feb. 27, 2025

Related U.S. Application Data
(60) Provisional application No. 63/406,023, filed on Sep. 13, 2022.

(51) Int. Cl. A61M 37/00

(52) U.S. Cl. CPC A61M 37/0015; A61M 2037/0023; A61M 2037/0046; A61M 2037/0061

(57) ABSTRACT

A device, or tool, for applying microneedles to skin or other biological tissue is provided, wherein the device includes an applicator which has an array of microneedles; and a trigger mechanism operably connected to the applicator. The device is configured to apply the array of microneedles to skin with a controlled velocity, force, and angle. The device has no stored energy, and the trigger mechanism does not permit the array of microneedles to be displaced toward to the skin until a threshold manual force applied to the device.

### CROSS-REFERENCE TO RELATED APPLICATIONS

This application claims priority to U.S. Provisional Application No. 63/406,023, filed Sep. 13, 2022, which is incorporated herein by reference.

### FEDERALLY SPONSORED RESEARCH AND DEVELOPMENT

This invention was made with government support under prime contract number 7200AA20CA00016, awarded by the U.S. Agency for International Development. The government has certain rights in the invention.

### BACKGROUND

Microneedle array devices are applied to biological tissue, such as skin, for various purposes, including but not limited to drug delivery (e.g., vaccinations, contraceptive agent administration) and diagnostics. The microneedles of the arrays generally should be applied to the tissue with a certain force, velocity, and insertion angle in order to achieve a desired penetration of the microneedles into the tissue. Insufficient insertion force, insufficient velocity, or a non-perpendicular insertion angle may undesirably produce incomplete or no insertion and/or inadvertent breaking of microneedle tips.

Conventional microneedle insertion may require user training to apply the right force, velocity, and insertion angle for successful application of a microneedle patch to a patient's skin, for example. Alternatively or in addition, complex and/or expensive applicators or application systems may be required. For example, some conventional systems rely on energy stored within a microneedle patch or an additional component associated with the patch to provide the microneedle insertion force. The energy may, for instance, be stored in the form of a spring or an elastic material, or it could be in the form of a battery that energizes a moving component that applies the patch to the tissue. However, the pre-stored energy may increase the risk that the microneedle array will be prematurely deployed, which may result in wasted materials and/or improper application. In addition, conventional tools with pre-stored energy may also require complex and specialized components, making them difficult and/or expensive to manufacture.

Self-administration of microneedle patches would improve user compliance and positively affect many pharmaceutical applications. However, given the importance described above that the microneedles be properly applied to skin or other tissue in order for the microneedles to be correctly inserted, user error must be minimized for self-administration. It therefore would be desirable to provide new and improved tools that facilitate reproducible and effective insertion that could increase the success rate of microneedle applications. Such tools desirably would be relatively simple and inexpensive to produce and easy to use for self-administration.

### BRIEF SUMMARY

In one aspect, a device, or tool, for applying microneedles to skin or other biological tissue is provided, wherein the device includes an applicator which comprises an array of microneedles; and a trigger mechanism operably connected to the applicator, wherein the device is configured to apply the array of microneedles to skin with a controlled velocity, force, and angle, and wherein the device has no stored energy and the trigger mechanism does not permit the array of microneedles to be displaced toward to the skin until a threshold manual force applied to the device.

In a particular embodiment, the device includes: an applicator portion which comprises (i) a support structure from which an array of microneedles extend, and (ii) a release mechanism; and a base portion configured to rest against the skin or other biological tissue and hold the applicator portion in a pre-launch position in which the microneedles face the skin or other biological tissue a spaced distance away from a surface of the skin or other biological tissue, wherein the applicator portion is configured to receive a manually applied force which, upon exceeding a predetermined threshold, causes the release mechanism to trigger and release the microneedles together with at least part of the support structure from the pre-launch position, thereby driving the microneedles along a guide path toward the skin or other biological tissue at a force and a velocity effective to insert the microneedles into the skin or other biological tissue. The release mechanism may be configured to trigger by mechanical fracture of a portion of the support structure, e.g., at a plurality of perforations or predefined lines of weakness in the support structure, or by deformation of a latch feature.

In another aspect, a method is provided that includes: positioning a base of an applicator tool against a target tissue surface wherein an array of microneedles extend from an applicator disposed in a pre-launch position within the base, with the microneedles facing the target tissue surface a spaced distance away therefrom by at least 5 mm; manually applying a force to an upper surface of the applicator tool to exceed a predetermined threshold, causing a release mechanism connecting the applicator to the base to trigger and release the microneedles together with at least part of the applicator, thereby driving, via the manually applied force, the microneedles along a guide path toward the tissue surface at a force and a velocity effective to insert the microneedles into the skin or other biological tissue, wherein the triggering of the release mechanism comprises (i) mechanical fracture of a structure securing the applicator in the pre-launch position, or (ii) deformation of a latch feature. The velocity may be between about 1 m/s to about 15 m/s, preferably about 8 m/s, and the spaced distance may be from 5 mm to 3 cm, preferably about 15 mm.

### BRIEF DESCRIPTION OF THE DRAWINGS

The detailed description is set forth with reference to the accompanying drawings.

The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1 is a perspective view of a microneedle patch, according to one embodiment of the present disclosure.

FIG. 2A is a perspective view of a microneedle applicator tool, according to one or more embodiments of the present disclosure.

FIG. 2B is a perspective view of the base portion of the microneedle applicator tool of FIG. 2A, according to one or more embodiments of the present disclosure.

FIG. 2C is an exploded perspective view of the applicator portion of the microneedle applicator tool of FIG. 2A, according to one or more embodiments of the present disclosure.

FIG. 3A is a perspective view of a microneedle applicator tool, according to one or more embodiments of the present disclosure.

FIG. 3B is a perspective view of the base portion of the microneedle applicator tool of FIG. 3A, according to one or more embodiments of the present disclosure.

FIG. 3C is an exploded perspective view of the application portion of the microneedle applicator tool of FIG. 3A, according to one or more embodiments of the present disclosure.

FIG. 4A is a perspective view of a microneedle applicator tool, according to one or more embodiments of the present disclosure.

FIG. 4B is a perspective view of the base portion of the microneedle applicator tool of FIG. 4A, according to one or more embodiments of the present disclosure.

FIG. 4C is a perspective view of the application portion of the microneedle applicator tool of FIG. 4A, according to one or more embodiments of the present disclosure.

FIG. 5 is a perspective view of a microneedle applicator tool, according to one or more embodiments of the present disclosure.

FIG. 6 is a perspective view of a microneedle applicator tool, according to one or more embodiments of the present disclosure.

FIG. 7 is a perspective view of a microneedle applicator tool, according to one or more embodiments of the present disclosure.

FIG. 8 is a perspective view of a microneedle applicator tool, according to one or more embodiments of the present disclosure.

FIG. 9A is a perspective view of a microneedle applicator tool for simultaneous application of multiple microneedle patches, according to one or more embodiments of the present disclosure.

FIG. 9B is a bottom plan view of a microneedle applicator tool for simultaneous application of multiple microneedle patches, according to one or more embodiments of the present disclosure.

FIG. 9C is a bottom perspective view of a microneedle applicator tool for simultaneous application of multiple microneedle patches, according to one or more embodiments of the present disclosure.

FIG. 9D is a bottom plan view of a microneedle applicator tool for simultaneous application of multiple microneedle patches, according to one or more embodiments of the present disclosure.

FIG. 10 is a perspective view of a microneedle applicator tool for sequential application of multiple microneedle patches, according to one or more embodiments of the present disclosure.

FIG. 11 is a partially transparent perspective view of a microneedle applicator tool for sheering application, according to one or more embodiments of the present disclosure.

FIG. 12A is a perspective view of a microneedle applicator tool, according to one or more embodiments of the present disclosure.

FIG. 12B is a side view of the microneedle applicator tool of FIG. 12A, according to one or more embodiments of the present disclosure.

FIG. 13 depicts insertion of a microneedle patch using a microneedle applicator tool, according to one or more embodiments of the present disclosure.

FIG. 14 is a representative bright-field image showing insertion of microneedles into an artificial skin model using a microneedle applicator tool, according to one or more embodiments of the present disclosure.

FIG. 15 is an optical coherence tomography (OCT) image showing insertion of microneedles into an artificial skin model using a microneedle applicator tool, according to one or more embodiments of the present disclosure.

### DETAILED DESCRIPTION

Microneedle application devices, systems, and methods have been developed for more easily and effectively applying microneedles to the skin, or other tissues for medical or other purposes. Factors such as the force, velocity, acceleration, and angle between the patch surface and the tissue all influence the interaction between the patch and the tissue, and importantly, influences whether the microneedles puncture the tissue and the depth to which the microneedles are inserted into the tissue. The presently disclosed applicator devices are configured to enable a user to apply a sufficient and evenly distributed insertion force to a microneedle array and predictably achieve a desired insertion velocity and insertion angle (e.g., perpendicular to the skin surface). Generally, the microneedle applicator tools disclosed herein store energy while the user is actively applying force to the tool, and upon building up and exceeding a threshold amount of energy, releasing said energy in a manner that is effective to apply the microneedles to the skin. If at any point the user stops applying the force, any stored energy will be released and no additional energy will be stored unless the user again applies force to the tool. That is, the tool is not capable of storing energy in the device while force is not being applied to the tool.

Unlike conventional microneedle applicator and patch designs, energy for application of the microneedle application tools described herein is solely supplied by a user's manually applied force (e.g., by the user's thumb) to the application tool. With the tools described herein, the application of the insertion force to the microneedles does not involve energy from a battery or separate spring elements. The device designs beneficially may be easier and more cost-effective to fabricate and use, and advantageously may enhance the reliability or effectiveness of self-administration. Advantageously, the microneedle application tools disclosed herein provide high-impact application with a high velocity, while also ensuring a controlled (e.g., perpendicular) insertion angle.

The success of microneedle application to skin or other tissues generally relies on several parameters. First, the sharpness of microneedle tips is essential. The currently disclosed applicators advantageously may shield and protect the microneedles until the moment of insertion. Second, microneedles generally should be inserted perpendicularly (although, in some cases, non-perpendicular insertion may be desirable), and an adequate amount of pressure should be uniformly distributed on the microneedle patch. Angled insertion may break the microneedles or produce an uneven or insufficient pressure which may impede insertion and drug delivery. The currently disclosed applicators advantageously promote uniform insertion of the microneedles at a controlled (e.g., perpendicular) angle.

An important part of the tools disclosed herein is having a space between the microneedles and the skin (before the applicator is launched) in order to be able to achieve a high enough velocity for insertion of the microneedles, e.g., a spaced distance of at least 5 mm.

### The Applicator Devices (Tools)

In some embodiments, a device is provided for applying microneedles to skin or other biological tissue, wherein the device includes (a) an applicator portion which comprises (i) a support structure from which an array of microneedles extend, and (ii) a release mechanism; and (b) a base portion configured to rest against the skin or other biological tissue and hold the applicator portion in a pre-launch position in which the microneedles face the skin or other biological tissue spaced a distance away from a surface of the skin or other biological tissue, wherein the applicator portion is configured to receive a manually applied force which, upon exceeding a predetermined threshold, causes the release mechanism to trigger and release the microneedles together with at least part of the support structure from the pre-launch position, thereby driving the microneedles along a guide path toward the skin or other biological tissue at a force and velocity effective to insert the microneedles into the skin or other biological tissue.

In some preferred embodiments, the device is configured to produce a velocity effective to insert the microneedles that is from 1 m/s to 15 m/s, preferably about 8 m/s. A lower velocity may permit the target tissue to be elastically deform in contact with the microneedles rather than be penetrated by them. The desired velocity may be achieved by design factors including the spacing between the microneedle tips in the pre-launch position and the skin surface, and the force applied to the microneedles at launch. For example, in some preferred embodiments, the predetermined threshold of the manually applied force is from 10 N to 70 N, preferably about 40 N.

Factors such as the microneedle geometry, number of microneedles, and/or the density of microneedles in the array may impact the optimal insertion force. The release mechanisms described herein may be modified to adjust the insertion force. For example, the insertion force may be adjusted by changing the material of construction, thickness and/or geometry of a release portion designed to fail by fracture or elastic or plastic deformation. For example, using a thinner or perforated material for the release portion may reduce the insertion force, whereas a thicker material for the release portion may increase the insertion force.

Triggering of the release mechanism releases the energy temporarily stored in the applicator device, transferred from the user's application of force to it. The released energy is effective to launch the applicator portion and insert the array of microneedles into the skin (or other biological tissue), without any additional energy source. That is, energy is only stored while the user applies force to the applicator device.

The base portion may include a stabilization portion configured to be placed against a patient's skin such that the guide path is perpendicular to a surface of the skin at a target site of insertion of the microneedles. The base portion may include one or more outer walls defining an opening in which the microneedles together with at least part of the support structure are configure to travel along the guide path. In some embodiments, the walls of the base portion include guiderails along which the applicator portion may translate. For instance the applicator portion may include slots that matingly engage with the guiderails. In some other embodiments, the applicator portion has the guiderails, and the base portion has the counterpart slots. In some embodiments, the opening defined by the one or more walls of the base portion is cylindrical and the applicator portion comprises an elongated cylindrical body configured to translate within the opening.

In some embodiments, the release mechanism is integral with the support structure.

The release mechanism may be configured to trigger by mechanical failure. In some embodiments, the release mechanism is configured to trigger by mechanical fracture of a portion of the support structure. The support structure may include a polymeric film, may include a plurality of perforations or predefined lines of weakness, or a combination thereof.

In some embodiments, the release mechanism includes a tape or other thin film structure designed to break when a force applied by a user exceeds the material's facture strength (ability to resist crack-based failure under an applied load) and release the energy stored therein. For instance, the applied force may deform the structure beyond its elastic limit, causing it to break and release the energy stored therein. In some embodiments, the structure includes weakened areas to more precisely control the insertion force and velocity of the microneedle patch. The weakened areas may be formed by reducing the thickness of the structure at particular areas and/or by including perforations or other defects to ensure that the structure fails at the desired area for uniform release.

The release mechanism may be configured to trigger by elastic or plastic deformation beyond a certain point, which result in an instantaneous displacement, without fracture, of a structural component holding the applicator portion in the pre-launch position and release of the energy stored therein. In some embodiments, the release mechanism is configured to trigger by elastic deformation of a latch feature. For example, the latch feature may include a lip (e.g., a ring, a tab, other protrusion, or the like) on the applicator portion that, in the pre-launch position, interferes with a ledge (e.g., another ring, tab, other protrusion, or the like) on a wall of the base portion.

In some embodiments, the release mechanism is separate from the support structure. For example, the applicator portion may include an elongated body having an upper end portion configured to receive a manually applied force and a lower end portion including the array of microneedles, where the release mechanism is disposed between the upper end portion and the lower end portion.

### Microneedle Arrays and Patches

Essentially any microneedle array, or microneedle patch, can be used with the presently disclosed applicators. In particular embodiments, the microneedles comprise an agent of interest to be administered into the skin or other biological tissue. For instance, the microneedle array of the present disclosure may be a patch comprising dissolvable microneedles for administration of a drug, as known in the art.

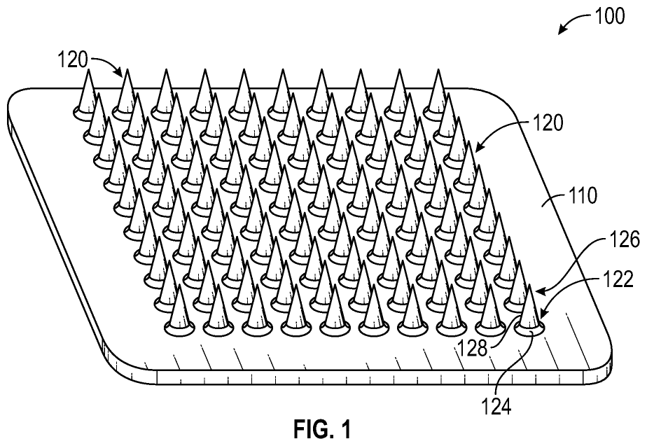

One example of a microneedle patch with an array of microneedles is depicted in FIG. 1. The microneedle patch 100 includes a backing layer 110 from which microneedles 120, in a 10×10 array, extend. The phrase “backing layer” and the terms “base substrate” or “substrate” are used interchangeably herein. Each microneedle 120 has a proximal end 122 attached to the backing layer 110 directly, or indirectly, via one or more proximal portions 124, and a distal tip end 126 which is sharp and effective to penetrate biological tissue. The microneedle 120 has tapered sidewalls 128 between the proximal end 122 and the distal end 126 of the microneedle 120.

A wide range of drugs may be formulated for delivery to biological tissue using microneedle patches. As used herein, the term “drug” refers to a prophylactic, therapeutic, or diagnostic agent useful in medical applications, as well as agents used in cosmetic, cosmeceutical, tattoo, or other non-medical applications. It may be any suitable active pharmaceutical ingredient or allergen. The drug may be a hormone, such as a contraceptive hormone, or a vaccine. Examples of vaccines include vaccines for infectious diseases, therapeutic vaccines for cancer, neurological disorders, allergies, and smoking cessation or other addictions.

In some embodiments, the structure and composition of the arrays of microneedles useful with the presently disclosed applicators are described in U.S. Pat. Nos. 10,265,511; 10,828,478; 10,940,301; 11,730,937; U.S.

8 Publication 2022/0401715; and WO 2023/164306, which are incorporated herein by reference.

### Specific Applicator Devices, Systems, and Methods

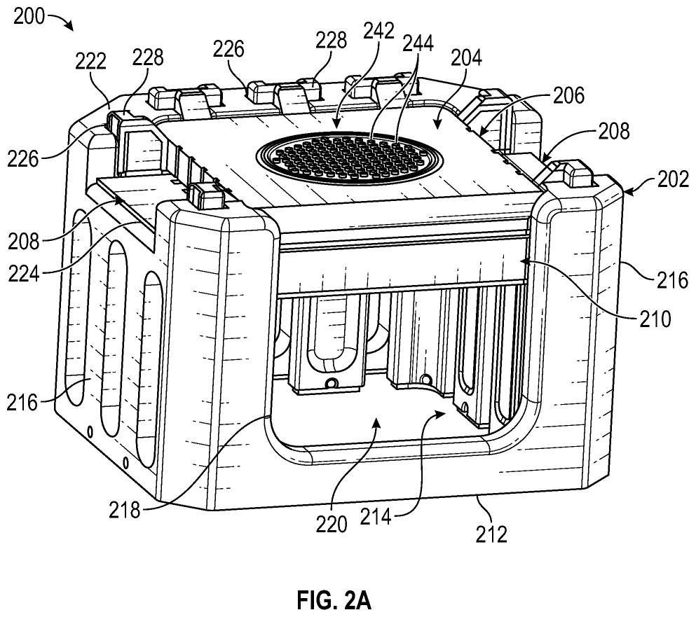

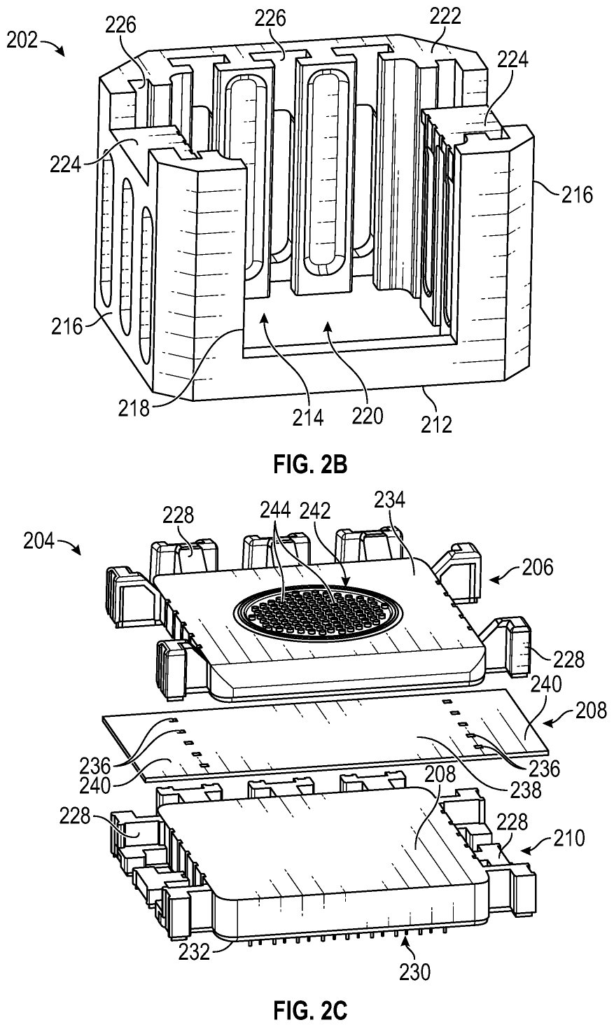

Embodiments of microneedle application tools and systems, and methods of use thereof, are shown in FIGS. 2A-13. Generally, the microneedle application tools include a base portion and an applicator portion (including an array of microneedles) operably connected thereto. The tool includes a release mechanism configured to fail upon manual application of a sufficient downward force onto the applicator portion, such that the failure launches the microneedles towards the patient's skin using only the manually applied force.

The microneedles are launched such that the microneedles penetrate the patient's skin at a desired, predetermined velocity. As used herein, “fail” or “failure” refers to fracture or deformation of the release mechanism.

FIGS. 2A-2C illustrate a microneedle application tool 200, which includes a base portion 202 and an applicator portion 204, which may be releasably attached to the base portion. The applicator portion 204 includes an upper portion 206, a release portion 208, and a backing portion 210. FIG. 2A shows the tool 200 with applicator portion in a pre-launch position in which the microneedles face the tissue surface spaced a distance away from the surface of the tissue.

The base portion 202 is configured to stabilize and orient the microneedle application tool 200 on a biological tissue, e.g., a patient's skin, during application of an array of microneedles. The foot 212 of the base portion 202, which is intended to be placed against the tissue surface (not shown) is shaped and dimensioned to provide the desired stabilization and orientation. The foot 212 has a substantially square shape, but may have any other suitable shape. The foot 212 has a bottom opening 214 through which the microneedles of the applicator portion 204 is guided in order to insert the microneedles into the tissue.

In some embodiments, at least a portion (i.e., one side) of the base portion 202 is open to accommodate variabilities in anatomy among potential users of the device to facilitate operation of the applicator tool. For example, keeping a portion of the tool open ensures that a user may easily orient and grasp the tool as needed to apply the necessary pressure to the applicator portion. In some cases, for instance, the thumb may be more easily inserted into the tool, while the user's other fingers may support the bottom of the base near the application area.

The base portion 202 has walls 216 that extend upward from the foot 212 of the base 202 to form a U-shape, defining a side opening 218 through which a user may place their thumb. The side opening 218 allows the thumb (i.e., the widest finger capable of generating the most force) to sit within the tool 200 and move freely until the array of microneedles is applied to the skin. The side opening 218 may also allow the user to maintain downward pressure on the applicator 204 while the applicator 204 passes through the internal cavity 220 of the base, without generating a rebounding force.

The base portion 202 has a top side 222 configured to hold at least a portion of the applicator portion 204 in a pre-launch position in which the microneedles face the tissue surface spaced a distance away from a surface of the tissue. The top side 222 of the base portion 202 has ledges 224 on opposing sides of the base 202 configured to hold at least a portion of the applicator portion 204, e.g., along opposing edge regions of part of the applicator portion. The ledges 224 are configured to hold a portion of the release portion 208 of the applicator 204, as shown in FIG. 2A.

The base portion 202 also includes slots 226 that are elongated in a direction perpendicular to the bottom surface of the foot. The slots 226 are positioned on sides of the walls 216 facing the cavity 220. Each wall 216 may have at least one slot 226. In the embodiment illustrated in FIGS. 2A-2C, one wall has exactly three slots, and two walls have exactly two slots. The slots 226 are configured to receive counterpart guiderails 228 that extend from sides of the applicator portion 204. The guiderails 228 are configured to slide vertically within the slots 226 so that the applicator portion 204 (and the array of microneedles extending therefrom) remains perpendicular to the tissue surface as the applicator portion translates toward the tissue surface. The slots and guiderails are dimensioned to limit tilting and/or wobbling of the applicator portion within the base during insertion of the microneedles, making the insertion process more precise and reproducible.

The slots 226 each have a T-shaped channel, although other channel shapes are possible; however, it is preferable to include a channel shape that is interlocking with the guiderails 228 to permits movement of the guiderails therein (and thus movement of the applicator portion) along only one axis with respect to the base portion. It is understood that the slot and guiderail features can be swapped, such that the base portion has the guiderails and the applicator portion has the slots.

As shown in FIG. 2A, the applicator portion 204 is generally sized and shaped to be disposed within cavity 220 of the base portion and to translate through the cavity. However, a part of a release portion 208 of the applicator portion extends laterally beyond the cavity 220 and contacts ledges 224 of base portion 202. This part of the release portion 208 of the applicator portion may be adhered or otherwise attached to the ledges 224 of the base portion 202, particularly if the release portion is a highly flexible material, such as a tape or polymeric film.

The applicator portion 204 may include an upper portion 206, a release portion 208, and backing portion 210. An array of microneedles 230 extend from the bottom side 232 of the backing portion 210. The microneedle array, e.g., a microneedle patch, may be releasably affixed to the bottom side 232 of the backing portion 210. The upper portion 206 include a user force application region 242 located about the center of the top surface 234 of the upper portion 206, which indicates the area to which a user should apply manual force, e.g., with a thumb, to initiate the microneedle application. The downward force should be applied by the user to the center of the upper portion 206 so that the release portion 208 fails evenly and the microneedle array is launched evenly toward the skin surface. While the user force application region 242 is depicted as circular, it is understood that other suitable shapes may be employed. In the illustrated embodiment, the user force application region 242 includes an anti-slip feature, which in this embodiment includes a plurality of nodules 244. In some other embodiments, the user force application region includes a concave area and/or includes ridges or texture to facilitate a user's grip. In some embodiments, the application region 242 may raised slightly from the top surface 234 of the upper portion 206 to further guide the user, for example in the form of a button (e.g., about 5 mm in height). Such anti-slip features may also help to prevent the user's thumb from shifting during use, thereby preventing stuttering or wobbling that could cause a failed application.

The applicator portion 204 is released from its pre-launch position shown in FIG. 2A upon a failure within the release portion 208. For example, the release portion 208 may include perforations 236, as shown in FIG. 2C, delineating the areas in which the release portion 208 is configured to fail. The perforations 236 may help to ensure that the release portion 208 is failing in the specified area. That is, the release portion 208 should fail along the perforations 236, such that the middle portion 238 of the release portion 208 remains with the rest of the applicator 204 during insertion, while the edge regions 240 of the release portion 208 remain affixed to the base 202. In other words, the fact that the edge regions 240 extend beyond the sides of the top portion 206 and backing portion 210, and are attached directly to the base 202, facilitates failure of the release portion along the perforations 236.

The microneedle application tool 200 may be constructed of any suitable materials. In some embodiments, the base 202 and upper portion 206 and backing portion 210 of the applicator 204 are constructed of the same material. In some embodiments, the base 202, the upper portion 206, and the backing portion 210 are constructed of two or more different materials. For example, the base 202, the upper portion 206, and/or the backing portion 210 may be constructed of plastic, metal, ceramic, glass, or any combination thereof. In some embodiments, the base 202, upper portion 206, and/or backing 210 may be constructed with recyclable and/or biodegradable materials, such as a recyclable and/or biodegradable plastic.

In some embodiments, the applicator of the microneedle application tool differs from that described with respect to FIGS. 2A-2C. For example, in some embodiments, the release portion is not separate from the upper portion and/or backing portions of the applicator. For example, the release mechanism may be incorporated into the upper portion or the backing portion of the applicator. In some embodiments, the applicator is a single piece that includes the release mechanism. In some embodiments, the applicator may be formed with the base of the microneedle application tool.

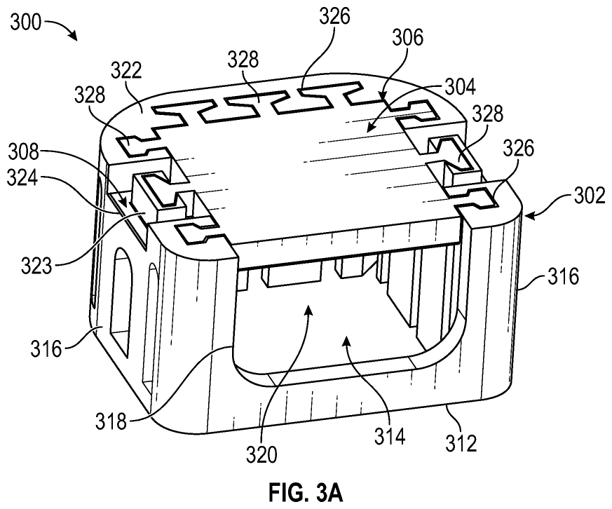

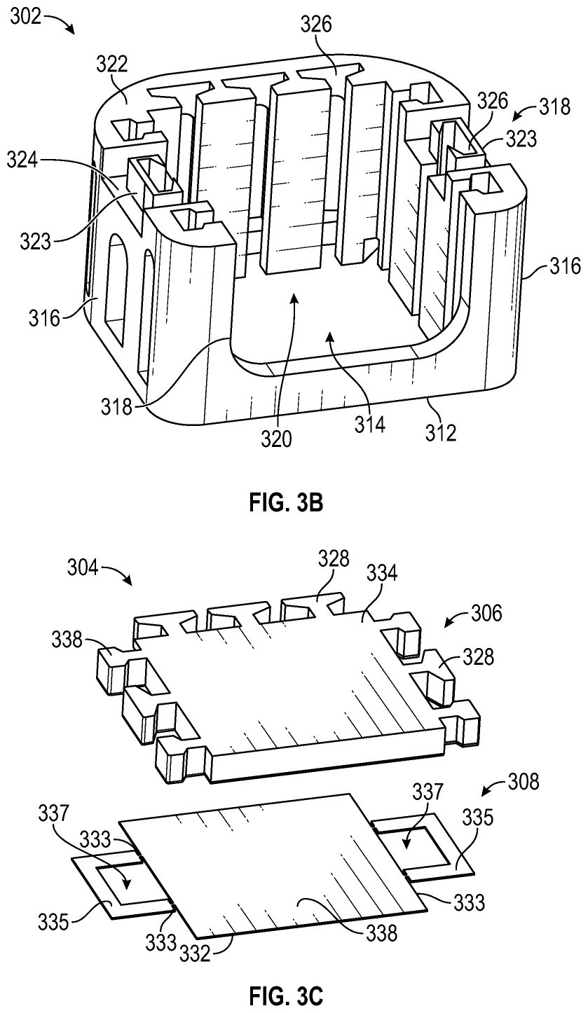

FIGS. 3A-3C illustrate a microneedle applicator tool 300 having a base portion 302 and an applicator portion 304. The applicator portion 304 includes an upper portion 306 and a backing portion 308. FIG. 3A shows the tool 300 with applicator portion in a pre-launch position in which the microneedles face the tissue surface spaced a distance away from a surface of the tissue.

The base portion 302 is similar to base portion 202 described with respect to FIGS. 2A-2C. For example, the foot 312 of the base portion 302 may be substantially square and define a bottom opening 314 through which the microneedles of applicator portion 304 pass to penetrate the skin. The base portion 302 has three walls 316 extending upward from the foot 312 of the base portion 302 to form a U-shape, thereby defining a side opening 318 through which a user may place their thumb in order to hold the applicator portion 304 while it passes through the internal cavity 320 defined by the walls 316.

The base portion 302 also includes a top side 322 with openings configured to receive the applicator portion 304. The top side 322 includes ledges 324 on opposing walls 316 of the base portion 302, which ledges are configured to receive opposing sides of backing portion 308. In the illustrated embodiment, each ledge 324 has an upwardly directed protrusion 323 that is configured to pass through an opening 337 in the backing portion 308 to secure the backing portion 308 to the base portion 302.

The base portion 302 includes slots 326 that are positioned on sides of the walls 316 facing the cavity 320. Each wall 316 may have at least one slot 326. In the embodiment illustrated in FIGS. 3A-3C, each wall has exactly three slots. The slots 226 are configured to receive counterpart guiderails 328 that extend laterally from sides of the upper portion 306. The guiderails 328 are configured to slide vertically within the slots 326.

The applicator portion 304 includes an upper portion 306 and a backing portion 308 that has an array of microneedles (not shown) extending from its bottom side 332.

As shown in FIG. 3C, the backing portion 308 includes weakened attachment points 333 connecting the side loops 335 and the middle portion 338 of the backing portion 308. The backing portion 308 is configured to fail at the attachment points 333, where the middle portion 338 of the backing portion 308 holding the microneedles remains with the applicator portion 304 during insertion, while the side loops 335 remain affixed to the ledges 324 of the base portion 302. The upper surface 334 of the upper portion 306 may include, although not shown, a user force application region that includes an anti-slip feature as described above for applicator tool 200.

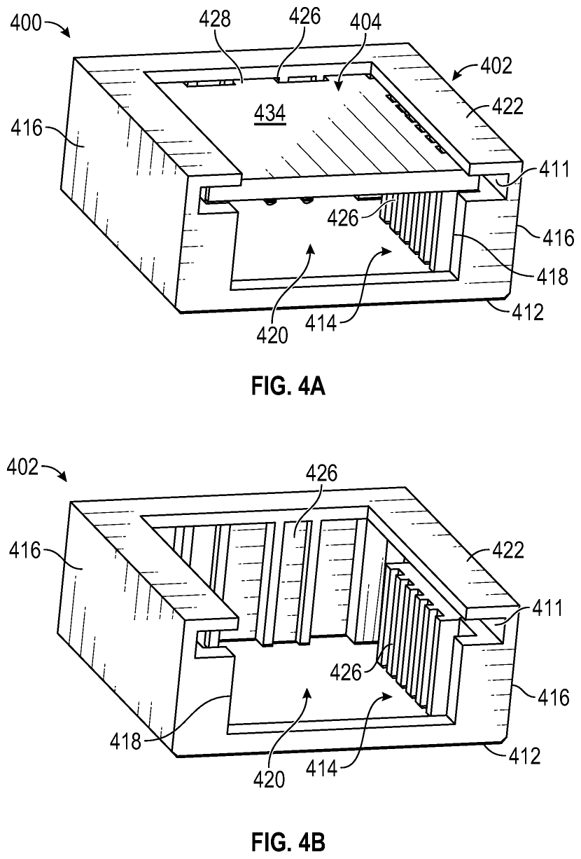

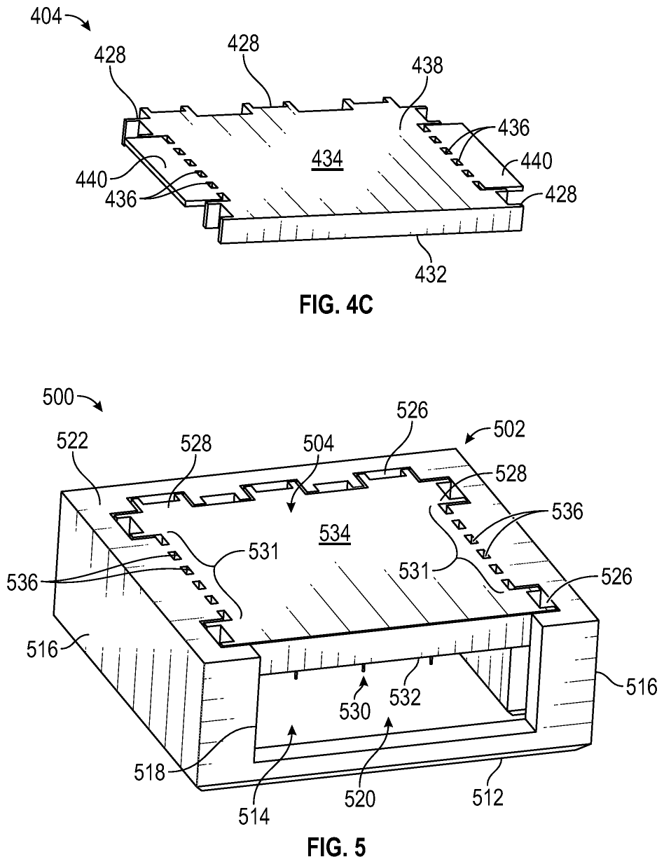

FIGS. 4A-4C illustrate another embodiment of a microneedle applicator tool 400 having a base portion 402 and an applicator portion 404. The applicator tool 400 differs from those shown in FIGS. 2A-3C in that the applicator portion 404 is a single component rather than an assembly of an upper portion and a release portion/backing portion.

Base portion 402 is similar to base portion 202 described with respect to FIGS. 2A-2C. The foot 412 of the base portion 402 is substantially square and defines a bottom opening 414 through which an array of microneedles extending from the applicator portion 404 may pass to apply the microneedles to the skin. The base portion 402 has three walls 416 extending upward from the foot 412 to form a U-shaped and defining a side opening 418 through which the user may place their thumb in order to apply pressure on the applicator 404 while the applicator 404 passes through the internal cavity 420 defined by the walls 416 of the base portion 402.

The base 402 also has a lateral slot 411 below the top side 422 of the base portion 402. The applicator portion can be slid horizontally into the lateral slot 411 to position the applicator portion in its pre-launch position.

The base 402 also includes slots 426 that are elongated in a direction perpendicular to the bottom surface of the foot. The slots 626 are positioned on sides of the walls 416 facing the cavity 420. The slots 426 are configured to receive counterpart guiderails 428 that extend from sides of the applicator portion 404. The guiderails 428 are configured to slide vertically within the slots 426 so that the applicator portion 204 (and the array of microneedles extending therefrom) remains perpendicular to the tissue surface as the applicator portion 404 translates toward the tissue surface.

The microneedle array (not shown) is attached to the bottom side 432 of the applicator portion 404. As shown in FIG. 4C, the applicator portion 404 has a main, central body region 438 disposed between two edge regions 440, and includes lines of perforations 436 between the main body portion 438 and the edge regions 430. The main body portion 438 is constructed, e.g., sufficiently rigid, to distribute the insertion force evenly across the microneedle array to facilitate uniform insertion of the microneedles. The perforations 436 facilitate mechanical failure at the desired positions between the main body portion 438 and the edge regions 440 when sufficient force is applied to the top surface 434 of the main body portion 438. In some other embodiments, the applicator portion may include other features e.g., weakened portions/area, in place of or in addition to the perforations, to control the failure force and location that produces the desired insertion velocity and force driving insertion of the microneedles.

Under a sufficient load, the applicator portion 404 is designed to fail along the perforations 436 so that the main portion 438 is launched toward the tissue surface with the microneedles to insert the microneedles, while the edge regions 440 remain disposed within the lateral slot 411.

FIG. 5 illustrates yet another embodiment of a microneedle applicator tool 500. In this tool, the base portion 502 and applicator portion 504 are formed together as a single piece, with the applicator portion in the pre-launch position. The base portion 502 is similar to base portion 202 as described above with respect to FIGS. 2A-2C. The foot 512 is substantially square and defines a bottom opening 520 through which the microneedles 530 extending from the bottom surface 532 of the applicator portion 504 may pass in order to apply the microneedles to the skin. The base 502 has three walls 516 extending upward from the foot 512 and form a U-shape side opening 518 through which a user may place their thumb in order to apply pressure on upper surface 534 of the applicator portion 504 while the applicator portion 504 passes through the internal cavity 514 defined by the walls 516 of the base portion 502. The interior sides of walls 516 include a plurality of slots 526 open toward on the cavity 514. The slots 526 are configured to receive counterpart guiderails 528 that extend from sides of the applicator portion 504. The guiderails 528 are configured to slide vertically within the slots 526 so that the applicator portion 504 (and the array of microneedles extending therefrom) remains perpendicular to the tissue surface as the applicator portion 504 translates toward the tissue surface.

At the upper side 522 of the base portion, the applicator portion 504 and the base portion are connected to one another through two weakened areas 531, which includes a line of perforations 536. Under a sufficient load applied downwardly on upper surface 534, the applicator portion 504 and base portion 502 separate (break) along the perforations 536 so that the applicator portion and microneedles are launched toward the tissue surface to insert the microneedles.

The microneedle application tools described herein generally are configured for applying the microneedles to a relatively flat area of skin of a limb or other body part of a human patient, although the tools readily can be adapted to other biological tissues, which may or may not be substantially flat. In some other embodiments, it would be useful to apply a microneedle patch to a thin, flexible tissue that can be held in a relatively flat orientation to receive a microneedle array. For example, in veterinary or animal husbandry applications, it may be useful to apply a microneedle patch to an ear of a dog, cat, cattle, pig, sheep, etc., for a variety of therapeutic, vaccination, identification, or tracking purposes.

Accordingly, the microneedle application tools described above may be adapted to further include a connected or separate tissue support structure that is configured to support and orient a flat tissue while a microneedle patch is applied.

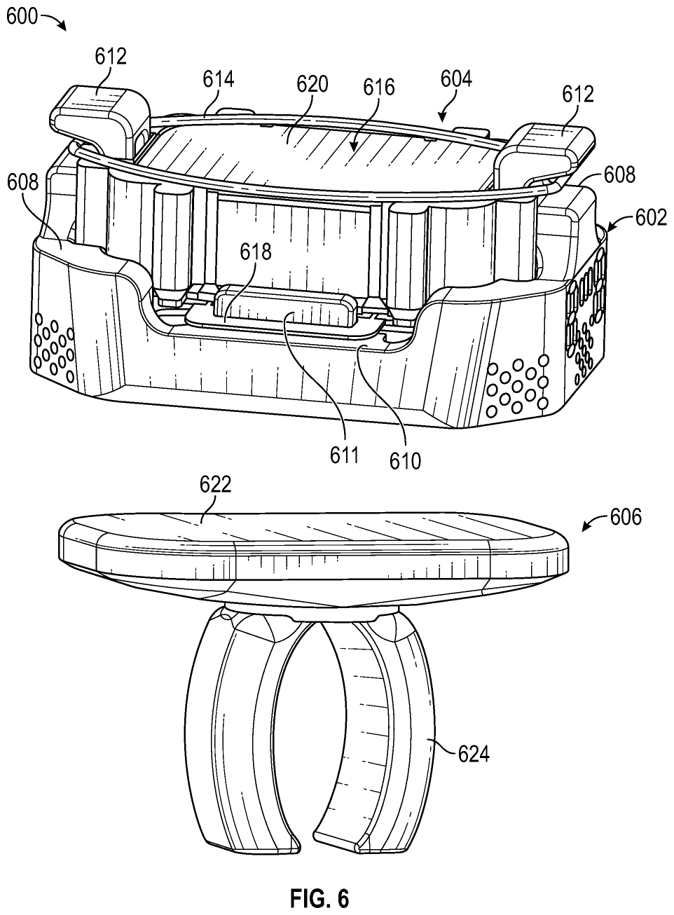

FIG. 6 illustrates a microneedle application tool 600 having a base 602, an applicator 604, and a tissue support portion 606. In general, the base 602 and applicator 604 may be similar in structure and function to those of the microneedle application tools previously described with respect to FIGS. 2A-5.

The base 602 is similar to the base portion 302 of FIGS. 3A-3C. The base 602 has a top side 608 configured to hold at least a portion of the applicator 604 thereon. The top side 608 of the base 602 includes ledges 610 on opposing sides of the base 602 configured to hold the applicator 604. The ledges 610 have upward projecting protrusions 611 for further securing the applicator 604 to the base 602. The base 302 may also include guiderails (not shown), which may be similar to those described with respect to any of the aforementioned embodiments. The base 602 also includes hooks 612 for attaching a strap 614 to the application tool 600. The strap 614 may be an elastic strap and may help to further steady the application tool 600 within a user's hand during its use. For example, a user may place their thumb (or other finger being used to depress the applicator 604) between the strap 614 and the applicator 604, helping to secure the application tool 600 to the user.

The applicator 604 is similar to the applicator portion 304 of FIGS. 3A-3C. The applicator 604 includes an upper portion 616 and a backing portion 618. The array of microneedles (not shown) may be located on the bottom side of the backing portion 618, such that when a sufficient downward force is applied by the user to the top side 620 of the top portion 616, the applicator 604 is released from the base 602 and the microneedles are applied to the skin.

In use, the base 602 and applicator 604 are placed on one side of a target tissue (e.g., the skin on an animal's ear) and the tissue support portion 606 is placed on an opposing side of the target tissue. The tissue support portion 606 includes a substantially planar platform 622 that is configured to contact the skin and a handle 624 configured for a user to hold and control the support portion. The platform 622 therefore is placed opposite the base 602 on the target tissue to effectively support the base 602 thereon to ensure proper insertion of the microneedles. In the illustrated embodiment, the handle 624 has a ring-like shape to enable the support portion to be worn on the user's finger to facilitate ease of use of the application tool 600. The ring may be adjustable to accommodate different fingers and/or different users.

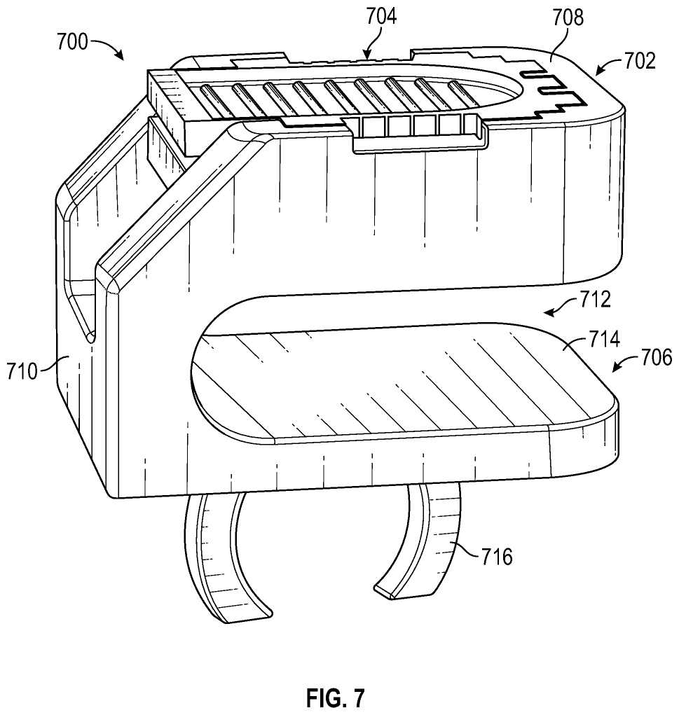

FIG. 7 illustrates another embodiment of a microneedle application tool 700 having a base 702, and applicator 704, and a tissue support portion 706. The base 702 and applicator 704 may be similar to the base portion 502 and applicator portion 504 of FIG. 5, respectively. The base 702 and the applicator 704 may be formed as a single piece, in which the applicator 704 is attached near the top side 708 of the base 702 via at one or more weakened areas that that are configured to fail upon application of a sufficient amount of force to the applicator 704. The base 702 and the tissue support portion 706 are connected by a connecting portion 710, which forms a gap 712 between the bottom of the base 702 and the platform 714 of the tissue support portion 706. To apply the microneedles using the microneedle application tool 700, the tissue is placed in the gap 712 so that it contacts at least the platform 714, and in some embodiments, the bottom of the base 702 and the platform 714. That is, the tissue may not initially contact the bottom of the base 702, but may contact the bottom of the applicator 704 as the applicator 704 travels towards the tissue. Thus, the connecting portion 712 of the tool 700 preferably is flexible or otherwise adjustable so that it can be configured to vary the height of the gap within a useful range of tissue thicknesses, so that the tissue can be securely in contact with the foot of the base while also being supported by tissue support portion 706. The tissue support portion also includes a handle 716, which may be a ring as described with respect to FIG. 6.

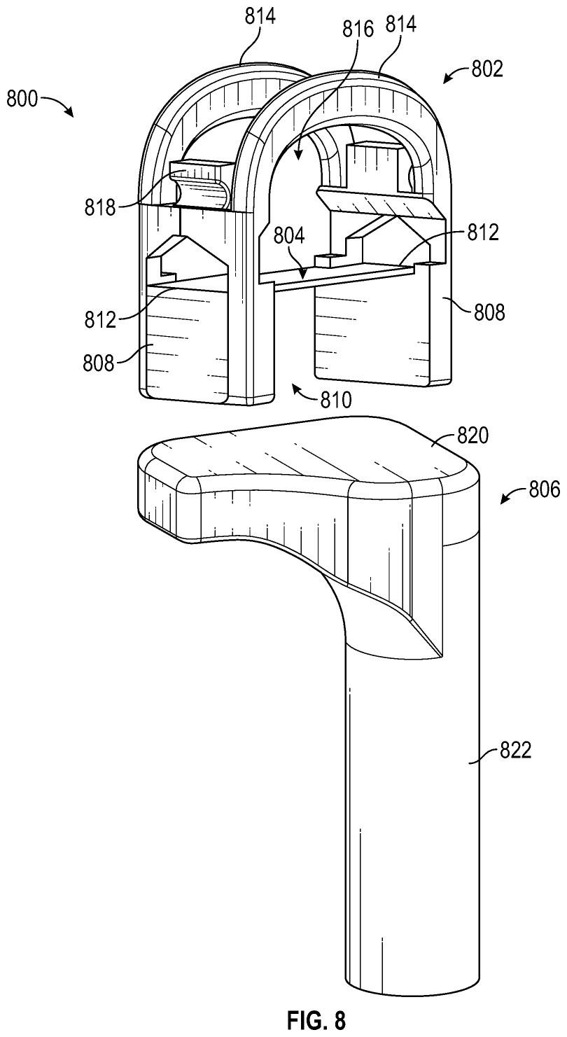

FIG. 8 illustrates another embodiment of a microneedle application tool 800, the tool 800 having a base 802, an applicator 804, and a tissue support portion 806. The base 802 has an arch shape and the applicator 802 has two legs 808 defining a bottom gap 810 therebetween through which the applicator 804 is to translate a microneedle array (not shown) toward and into a tissue. The applicator 804 is disposed at an intermediate position 812 between the legs 808 and the top of the arch 814. The applicator 804 includes weakened portions (not shown) where the applicator 804 is attached to the base 802. The weakened portions are configured to mechanically fail in order to apply the array of microneedles to the tissue, like described above with other embodiments.

An upper opening 816 is defined between the applicator 804 and the arch 814, in which the user may put their thumb (or another finger) in order to apply an insertion force to the applicator 804. The base 802 includes hooks 818 above the applicator 804, which may be used to secure a strap (not shown) to the base 802 to accommodate users with different anatomical characteristics.

In use, the base 802 and applicator 804 are placed one side of a target tissue (e.g., the skin on an animal's ear) and the tissue support portion 806 is placed on an opposing side of the target tissue. The tissue support portion 806 includes a substantially planar platform 820 configured to contact the target tissue and a handle 822 extending below the platform for a user to hold and control of the support portion. The platform 820 can be placed opposite the base 802 on the tissue to effectively support the base 802 thereon to ensure proper insertion of the microneedles. In some embodiments, the top of the platform 820 is covered or coated with a material to prevent the tissue from slipping and/or to provide additional comfort while the microneedles are being applied.

The microneedle applicator tool may be configured to apply multiple microneedle patches with a single applicator. In some embodiments, the microneedle applicator tool is configured to simultaneously apply multiple microneedle patches. In some embodiments, the microneedle applicator tool is configured to apply multiple microneedle patches in series. In some such embodiments, the applicator includes a top portion, a release portion, and a backing portion. In some other such embodiments, the applicator includes a top portion and a backing portion. In yet some other such embodiments, the applicator is a single piece. In still some other such embodiments, the applicator is formed as a single piece with the base.

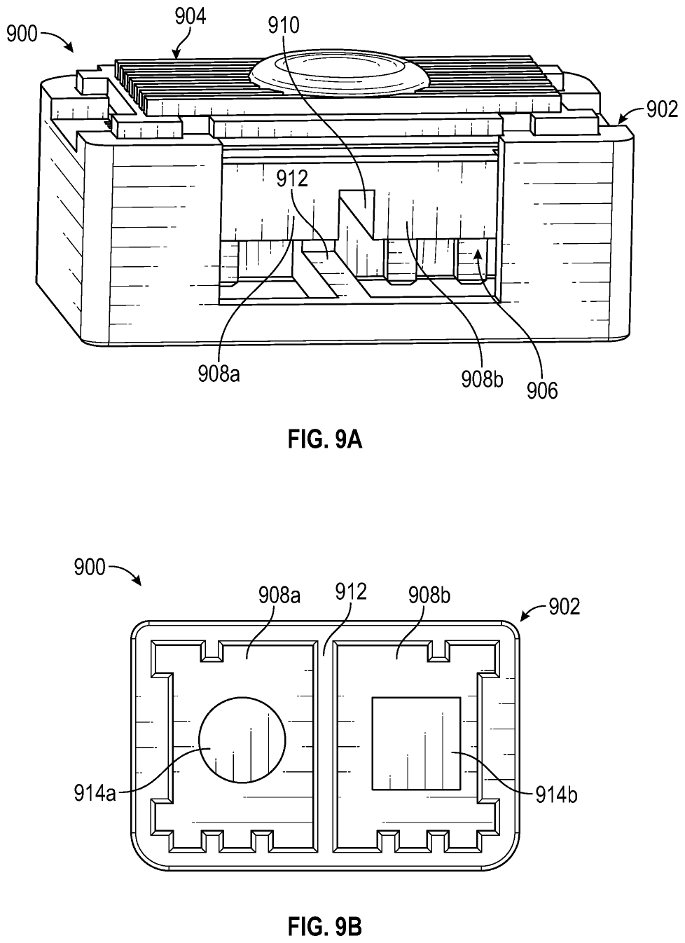

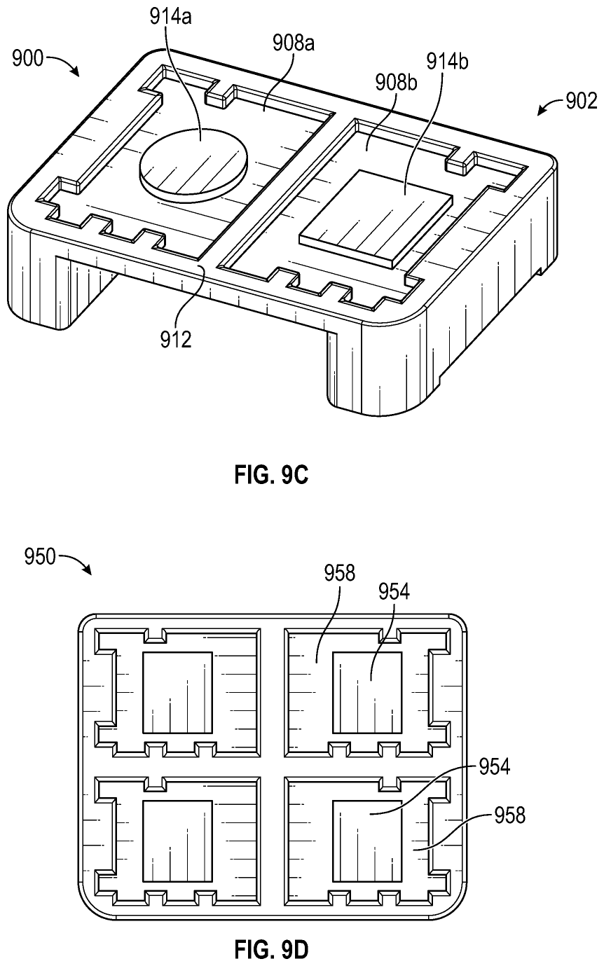

FIGS. 9A-9C illustrate a microneedle application tool 900 configured to simultaneously apply two microneedle patches. The microneedle application tool 900 includes a base 902 and an applicator 904. The base 902 and applicator 904 may be similar to the base portions and applicator portions described with respect to any of FIGS. 2A-5C.

As shown in FIG. 9B, the bottom side 906 of the applicator 904 is separated into a plurality of distinct blocks 908a and 908b onto which the microneedle arrays 914a and 914b are attached, respectively. The blocks 908a, 908b are separated by gap 910 which configured to receive a counterpart stopper 912 of the base 902 when the applicator is launched/microneedles inserted. Because the base 902 may have a larger profile to accommodate multiple microneedle patches, the stopper 912 may also provide the base 902 with added stability.

The microneedle application tool 900 is configured to administer two distinct microneedle arrays. The applicator 904 has two blocks 908a and 908b and is configured to simultaneously administer two microneedle arrays 914a and 914b. The applicator tool may be adapted to administer other numbers of microneedle patches and arrays, such as 3, 4, 5, 6, 7, 8, 9, or 10 (or more) microneedle arrays. For example, FIG. 9C shows applicator 950 which has four blocks 958 and is configured to simultaneously administer four microneedle arrays 954.

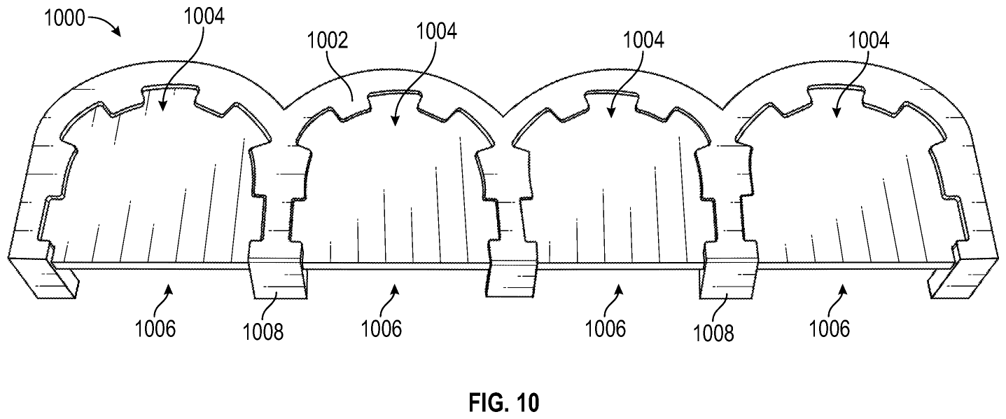

FIG. 10 illustrates one embodiment of a microneedle application tool 1000 configured to apply multiple arrays of microneedles in series. The microneedle application tool 1000 includes a base 1002 with multiple applicators 1004. The base 1002 and applicator 1004 may be similar to the base portions and applicator portions described with respect to any of FIGS. 2A-5C.

The base 1002 includes a plurality of openings 1006 for receiving applicators 1004 therein. Adjacent openings may be separated by a leg 1008 to maintain separation between the microneedle arrays.

The base 1002 may have any suitable number of openings dependent on the number of microneedle arrays to be administered. For example, the microneedle application tool 1000 may be configured to apply 2, 3, 4, 5, 6, 7, 8, 9, or 10 or more microneedle arrays. In the embodiment shown in FIG. 10, the microneedle application tool 1000 has four applicators 1004.

In certain embodiments, the microneedle application tool may be configured to insert an array of microneedles into the skin and apply a shear force, for example, to facilitate microneedle separation from patch substrate.

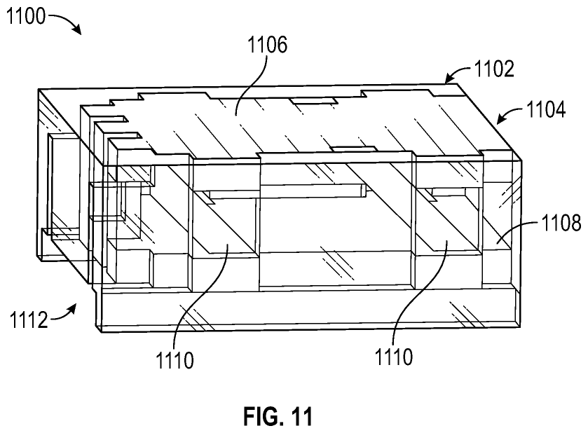

FIG. 11 illustrates a microneedle application tool 1100 for inserting a microneedle array with a sheering force, where the application tool 1100 includes a base 1102 and an applicator 1104. The base 1102 and applicator 1104 may be similar to those described with respect to any of FIGS. 2A-5C. In some embodiments, the applicator includes a top portion, a release portion, and a backing portion. In some embodiments, the applicator includes a top portion and a backing portion. In some embodiments, the applicator is a single piece. In some embodiments, the applicator is formed as a single piece with the base. The base 1102 is configured to support the applicator 1104, which has a top portion 1106 and a bottom portion 1108 connected by angled guiderails 1110. The base 1102 may also define a gap 1112 between the bottom portion 1108 and the skin, where the applicator 1104 may pass through the gap 1112 to apply the microneedles to the skin. The top 1106 of the applicator 1104 includes a predefined weakened portion configured to fail upon application of a sufficient amount of downward force to the applicator 1104. When the weakened portion fails, the guiderails 1110 may enable the applicator to move along the y-axis and then subsequently along the x-axis to apply the microneedles to the skin (e.g., an L-shaped motion). That is, the applicator 1104 first travels vertically to perpendicularly insert the microneedles into the tissue, then will begin to travel horizontally to apply a sheering force to the microneedles, thereby separating the microneedles from the backing.

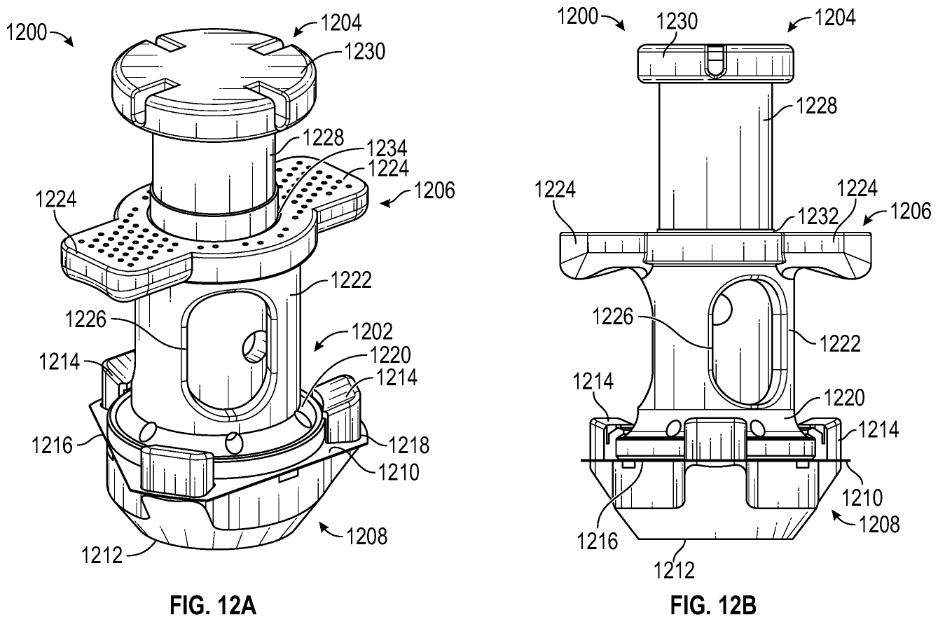

As shown in FIGS. 12A-12B, a microneedle application tool 1200 for applying an array of microneedles to biological tissue includes a base 1202 and an applicator 1204, where the base 1202 includes a guide portion 1206, a stabilization portion 1208, and a microneedle support portion 1210 disposed therebetween. The base 1202, and more specifically, the stabilization portion 1208, is configured to stabilize and orient the microneedle application tool 1200 on the biological tissue, e.g., a patient's skin, during application of an array of microneedles. The foot 1212 of the stabilization portion 1208 of the base 1202 may be substantially circular and defines a bottom opening (not shown) through which the applicator 1204 may pass in order to apply the microneedles to the skin. While the foot 1212 of the base 1202 is shown as being circular, it would be understood that other geometries are possible and contemplated by this disclosure. For example, the foot of the base could have any shape, such as a square or a rectangle.

The stabilization portion 1208 may extend at an angle upward from the foot 1212, and have at least one protrusion 1214 extending upward from the top surface 1216 of the stabilization portion 1208. The protrusions 1214 may be configured to receive and hold the microneedle support portion 1210 through reciprocal slots 1218 therein. The protrusions 1214 may also define a space therein configured to receive the guide portion 1206 of the base 1202, such that the guide portion 1206 may rest on top of the microneedle support portion 1210.

The guide portion 1206 is substantially cylindrical, with a bottom portion 1220 positioned atop the stabilization portion 1208 and microneedle support portion 1210, a cylindrical middle portion 1222 extending upward from the bottom portion 1220, and a lateral arm 1224 positioned at the top of the middle portion 1222. The lateral arm 1224 may help the user to hold the applicator tool 1200 during use. The middle portion 1222 includes a window 1226 through which the applicator 1204 may be visible. The window 1226 may also advantageously allow air between the applicator 1204 and the guide portion 1206 to escape as the applicator 1204 travels within the guide 1206, mitigating any negative effects compressed air may have on the application force and/or velocity. In use, the applicator 1204 is configured to apply the array of microneedles to the patient's skin. The applicator 1204 may include an elongate body 1228 slidably disposed within the guide portion 1206, and a stopper 1230 configured to abut the lateral arm 1224 of the guide portion 1206 when the applicator 1204 has successfully applied the array of microneedles to the skin.

The applicator 1204 includes a lip 1232 disposed around the circumference of the elongate body 1228. The lip 1232 is configured to rest on, or within, a reciprocal ledge 1234 of the guide portion 1206, the ledge 1234 defining the opening through which the applicator 1204 is received. Upon application of a downward force to the stopper 1230, the lip 1232 may begin to deform the ledge 1234. With continued force, the lip 1232 may deform the ledge 1234 to a point that the ledge 1234 will release the lip 1232, thereby releasing the applicator 1204 to apply the microneedles to the skin. In some alternative embodiments, the lip rather than the ledge may undergo the deformation triggering release. In various embodiments, the deformation of the lip and ledge may be elastic, plastic, or a combination thereof.

### Methods of Application

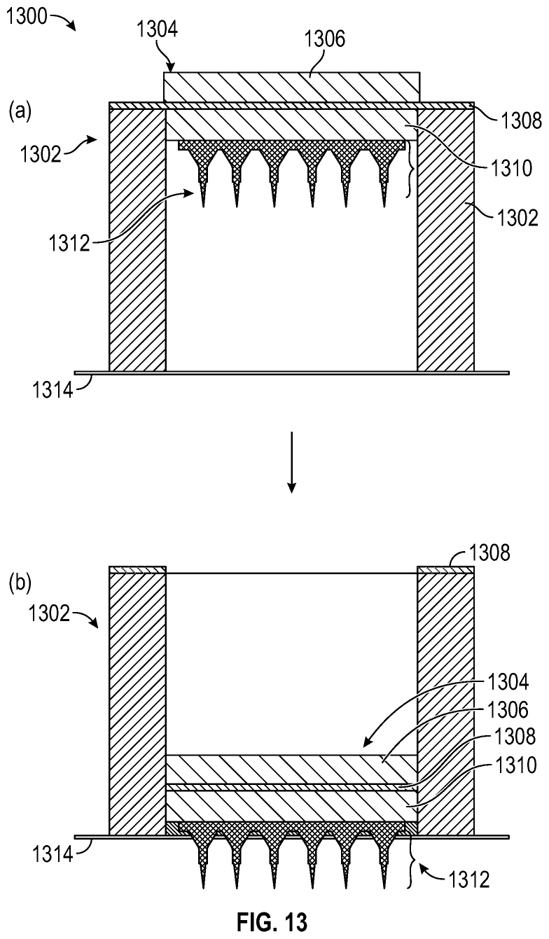

An exemplary method of using the microneedle tools and systems disclosed herein is illustrated in FIG. 13 Generally, the method includes placing an application tool with a base and an applicator portion on the target tissue and applying an effective amount of downward force to the applicator portion in order to release the applicator from the base. When the applicator is released from the base, the applicator will travel towards the target tissue to achieve a velocity sufficient to apply the microneedles to the target tissue. The factors impacting the velocity include the amount of energy stored in the tool from the user-applied force before release of the applicator is triggered and the distance between the microneedles and the tissue surface at before the applicator is released. In particular embodiments, the applicator is designed to produce a selected velocity profile, to provide a maximum velocity during contact/insertion of the microneedles.

As shown in FIG. 13, the microneedle application tool 1300 has a base 1302 and an applicator 1304, where the applicator 1304 includes a top portion 1306, a release portion 1308, and a backing portion 1310 to which a microneedle array 1312 is attached. As shown in step (a), the base 1302 of the microneedle application tool 1300 is placed onto the target tissue surface 1314, such that the longitudinal axes of the microneedles 1312 are substantially perpendicular to the tissue surface 1314, with the tip ends of the microneedles pointed toward, and spaced away from, the tissue surface.

After the microneedle application tool 1300 is properly placed on the target tissue surface 1314, a user manually applies a force to the top portion 1306 of the applicator 1304 until the release portion 1308 fails and releases the applicator 1304 from its pre-launch position within the base. The release portion 1308 is configured to mechanical fail in response to a sufficient user applied force. For example, the release portion 1308 may be formed of a material that may be stretched or strained to a threshold point, upon which the material will fracture (i.e., fail). In some embodiments, the force required for failure of the release portion 1308 is between about 10 N to about 70 N, such as between about 10 N to 60 N, about 20 N to about 50 N, about 30 N to about 50 N, or about 40 N.

When the applicator 1304 is released from the base 1302, the applicator 1304 travels downward to the tissue surface and perpendicularly inserts the microneedle array 1312 into the tissue 1314. The distance that the applicator 1304 travels depends on the height of the base 1302 and the height of the microneedle array 1312 above the tissue 1314 when the application tool 1300 is assembled prior to use. In some embodiments, this height is from about 5 mm to about 3 cm, such as from about 10 mm to about 20 cm, from about 5 mm to about 15 mm, or about 15 mm.

The velocity at which the applicator 1304 moves towards the tissue 1314 may be proportional to the force applied to the applicator 1304 to induce failure in the release portion 1308 and/or the distance the applicator 1304 travels to apply the microneedle array 1312 to the tissue. For example, a higher failure force may result in the microneedles 1312 being applied with a higher insertion velocity. In some embodiments, the velocity of the microneedle array 1312 is between about 1 m/s to about 15 m/s, such as between about 2 m/s to about 12 m/s, about 3 m/s to about 10 m/s, about 4 m/s to about 9 m/s, about 5 m/s to about 8 m/s, or about 8 m/s.

### EXAMPLES The invention can be further understood with reference to the following non-limiting examples.

### Example 1. Fabrication of Microneedle Patches

Microneedle arrays were fabricated using a solvent casting method. 90 μL of a water-based solution consisting of 18% PVA and 18% sucrose (w/w) was cast on microneedle molds and dried for 24 hours at room temperature. Upon demolding, the microneedle (array) patches were stored in a desiccator for at least 24 hours for further drying.

### Example 2. Fabrication and Assembly of Application Tools

Prototypes and test models were designed with CAD software (Fusion 360, Autodesk) and 3D printed with a fused deposition modeling-based 3D printer (Pro2 Plus, Raise3D) and a stereolithography-based 3D printer (Form3B+, Formlabs). Most of the pieces were made with polylactic acid (PLA), a recyclable 3D printing material, and/or a rigid resin.

An FDM (fused deposition modeling) printer was used for the initial fabrication with lower resolutions (high layer heights, e.g., 0.25 mm) to have an overall idea of the prototypes. Then, chosen candidate prototypes were printed with a higher resolution (lower layer heights, e.g., 0.05 mm) with a FDM or a SLA (stereolithography) printer. Once the pieces were printed, previously fabricated microneedle patches were attached to the microneedle support. Then, (i) the microneedle patch carrier and the surrounding part were coupled with an adhesive material (e.g., tape), or (ii) the microneedle support with rigid side parts was placed on top of the surrounding part. In the former embodiments, a rigid portion was placed on top of the adhesive material to prevent loosening, which would reduce the impact force and velocity of the microneedle patch upon insertion.

### Example 3. Insertion of Microneedle Patches into Artificial Skin Models and Cadaver Porcine Skin

Fabricated microneedle patches were fixed to the carrier part of the application tool with a double-sided tape or an adhesive spray. Next, the carrier with the microneedle patch was assembled with the remaining parts of the application tool. This was followed by alignment on an artificial skin model or cadaver porcine skin. The last step was applying enough force to break the release mechanism, move the microneedles rapidly to the skin model, and provide insertion.

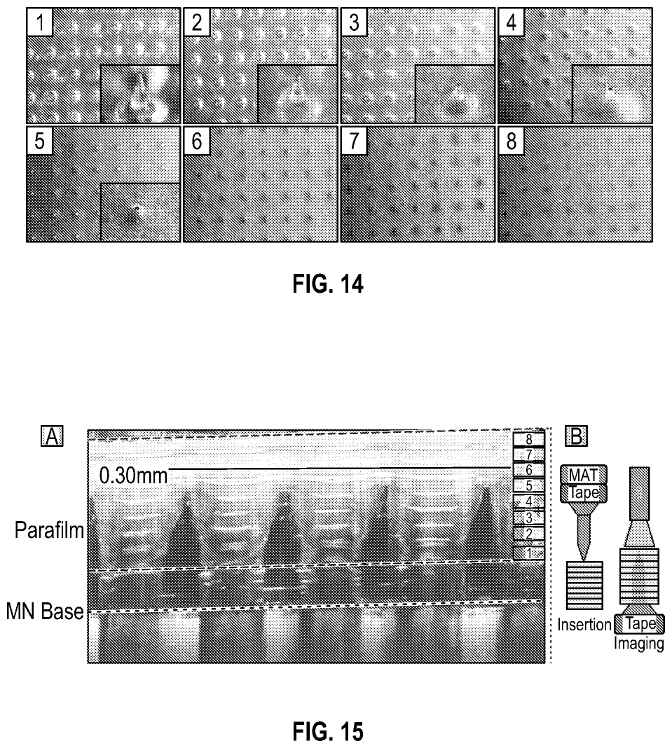

FIG. 14 shows representative bright-field images of the patch after it was applied to the artificial skin model. The skin model comprises eight layers of semi-transparent flexible film (Parafilm™). The images show that microneedles successfully perforated the skin model Parafilm layers.

### Example 4. Evaluation of Insertion Success and Insertion Depth

An optical microscope was used to evaluate the insertion depth by peeling the layers of the artificial skin model and inspecting each layer and the overall image of the cadaver porcine skin after insertion. An optical coherence tomography device was used to check the insertion depth while the microneedles were inside the artificial skin model.

FIG. 15 is an optical coherence tomography (OCT) image (A) showing insertion of the microneedles in an artificial skin model. The lines on the top and middle show the artificial skin model. The numbers on the right show the number of layers. The schematic representation of the imaging method is shown on the right side (B).

### EMBODIMENTS

Some embodiments of the present disclosure can be described in view of one or more of the following:

Embodiment 1. A device for applying microneedles to skin or other biological tissue, the device comprising: an applicator portion which comprises (i) a support structure from which an array of microneedles extend, and (ii) a release mechanism; and a base portion configured to rest against the skin or other biological tissue and hold the applicator portion in a pre-launch position in which the microneedles face the skin or other biological tissue a spaced distance away from a surface of the skin or other biological tissue, wherein the applicator portion is configured to receive a manually applied force which, upon exceeding a predetermined threshold, causes the release mechanism to trigger and release the microneedles together with at least part of the support structure from the pre-launch position, thereby driving the microneedles along a guide path toward the skin or other biological tissue at a force and a velocity effective to insert the microneedles into the skin or other biological tissue.

Embodiment 2. The device of Embodiment 1, wherein the base portion comprises a stabilization portion configured to be placed against a patient's skin such that the guide path is perpendicular to a surface of the skin at a target site of insertion of the microneedles.

Embodiment 3. The device of Embodiment 2 or 3, wherein the base portion comprises one or more outer walls defining an opening in which the microneedles together with at least part of the support structure are configured to travel along the guide path.

Embodiment 4. The device of any one of Embodiments 1 to 3, wherein the one or more outer walls comprises one or more guiderails or slots that matingly engage with one or more slots or guiderails of the applicator portion, respectively.

Embodiment 5. The device of any one of Embodiments 1 to 4, wherein the opening defined by the one or more walls of the base portion is cylindrical and the applicator portion comprises an elongated cylindrical body configured to translate within the opening.

Embodiment 6. The device of any one of Embodiments 1 to 5, wherein the release mechanism is configured to trigger by mechanical fracture of a portion of the support structure.

Embodiment 7. The device of any one of Embodiments 1 to 6, wherein the support structure comprises a polymeric film.

Embodiment 8. The device of any one of Embodiments 1 to 7, wherein the release mechanism comprises a plurality of perforations or predefined lines of weakness in the support structure.

Embodiment 9. The device of any one of Embodiments 1 to 5, wherein the release mechanism is configured to trigger by deformation of a latch feature.

Embodiment 10. The device of Embodiment 9, wherein the latch feature comprises a lip on the applicator portion that, in the pre-launch position, interferes with a ledge on a wall of the base portion.

Embodiment 11. The device of any one of Embodiments 1 to 10, wherein the release mechanism is integral with the support structure.

Embodiment 12. The device of any one of Embodiments 1 to 10, wherein the release mechanism is separate from the support structure.

Embodiment 13. The device of Embodiment 12, wherein the applicator portion comprises an elongated body having an upper end portion configured to receive a manually applied force and a lower end portion comprising the array of microneedles, where the release mechanism is disposed between the upper end portion and the lower end portion.

Embodiment 14. The device of any one of Embodiments 1 to 13, which is configured to produce a velocity effective to insert the microneedles that is from 1 m/s to 15 m/s, preferably about 8 m/s.

Embodiment 15. The device of any one of Embodiments 1 to 14, wherein the predetermined threshold of the manually applied force is from 10 N to 70 N, preferably about 40 N.

Embodiment 16. The device of any one of Embodiments 1 to 15, wherein the device is configured to apply more than one array of microneedles.

Embodiment 17. The device of any one of Embodiments 1 to 15, wherein the device is configured to apply more than one array or microneedles simultaneously.

Embodiment 18. The device of any one of Embodiments 1 to 15, wherein the device is configured to apply more than one array or microneedles in series.

Embodiment 19. A system comprising: an insertion tool comprising: a housing; and a backing releasably attached to the housing; and an array of microneedles comprising a substance of interest, wherein the array of microneedles is provided on a bottom side of the backing; wherein a user-applied force to the backing is effective to separate the backing from the housing and to insert the array of microneedles to a biological tissue at a predetermined velocity.

Embodiment 20. The system of Embodiment 19, wherein the predetermined velocity is between from about 1 m/s to 15 m/s, preferably about 8 m/s.

Embodiment 21. The system of Embodiment 19 or 20, wherein the user applied force is between from about 10 N to about 70 N, preferably about 40 N.

Embodiment 22. The system of any one of Embodiments 19 to 21, wherein the backing comprises: a top portion onto which the user-applied force is applied; an intermediate portion configured to releasably attach the backing to the housing; and a bottom portion, wherein the array of microneedles is provided on a bottom side of the bottom portion of the backing.

Embodiment 23. The system of Embodiment 22, wherein the intermediate portion comprises a tape or film configured to fail upon application of the user-applied force, thereby triggering said separation and insertion.

Embodiment 24. The system of Embodiment 22 or 23, wherein the intermediate portion comprises a predefined fracture region configured to fail upon application of the user-applied force to the backing, thereby triggering said separation and insertion.

Embodiment 25. The system of any one of Embodiments 19 to 21, wherein the backing comprises: a top portion onto which the user applied force is applied; and a bottom portion comprising a predefined fracture region configured to fail upon application of the user-applied force to the backing, thereby triggering said separation and insertion.

Embodiment 26. The system of any one of Embodiments 19 to 21, wherein the backing comprises a predefined fracture region configured to fail upon application of the user-applied force to the backing, thereby triggering said separation and insertion.

Embodiment 27. The system of any one of Embodiments 19 to 21, wherein the backing is releasably attached to the housing via a predefined fracture region configured to fail upon application of the user-applied force to the backing, thereby triggering said separation and insertion.

Embodiment 28. The system of any one of Embodiments 19 to 27, further comprising a tissue support portion configured to support the biological tissue from a side of the tissue opposing the user-applied force.

Embodiment 29. The system of Embodiment 28, wherein the tissue support portion is connected to the housing and defines a gap therebetween to receive the biological tissue.

Embodiment 30. The system of any one of Embodiments 19 to 27, wherein the backing is releasably attached to the housing via a latch mechanism and the user-applied force causes a mechanical deformation to separate the backing from the housing and to insert the array of microneedles.

Embodiment 31. A method comprising: positioning a base of an applicator tool against a target tissue surface wherein an array of microneedles extend from an applicator disposed in a pre-launch position within the base, with the microneedles facing the target tissue surface a spaced distance away therefrom by at least 5 mm; manually applying a force to an upper surface of the applicator tool to exceed a predetermined threshold, causing a release mechanism connecting the applicator to the base to trigger and release the microneedles together with at least part of the applicator, thereby driving, via the manually applied force, the microneedles along a guide path toward the tissue surface at a force and a velocity effective to insert the microneedles into the skin or other biological tissue, wherein the triggering of the release mechanism comprises (i) mechanical fracture of a structure securing the applicator in the pre-launch position, or (ii) deformation of a latch feature.

Embodiment 32. The method of Embodiment 31, wherein the velocity is between about 1 m/s to about 15 m/s, preferably about 8 m/s.

Embodiment 33. The method of Embodiment 31 or 32, wherein the spaced distance is from 5 mm to 3 cm, preferably about 15 mm.

Embodiment 34. The method of any one of claims 31 to 33, wherein the release mechanism connecting the applicator to the base comprises a tape.

Embodiment 35. The method of any one of Embodiments 31 to 34, wherein the release mechanism connecting the applicator to the base comprises the applicator being held in contact with the base, without being adhered or integrally connected, by forces incidental to the arrangement and dimensions of the applicator and base.

Embodiment 36. A device for applying microneedles to skin or other biological tissue, the device comprising: an applicator which comprises an array of microneedles; and a trigger mechanism operably connected to the applicator, wherein the device is configured to apply the array of microneedles to skin with a controlled velocity, force, and angle, and wherein the device has no stored energy and the trigger mechanism does not permit the array of microneedles to be displaced toward to the skin until a threshold manual force applied to the device.

Embodiment 37. The device of Embodiment 36, further comprising a base connected to the applicator and trigger mechanism and configured to be placed against a skin surface and stabilize a position of the applicator relative to the skin until the threshold manual force is applied to the device.

Embodiment 38. The device of Embodiment 36 or 37, wherein in the position the microneedles are spaced a distance away from the skin.

Embodiment 39. The device of any one of Embodiments 36 to 38, wherein the distance is from about 5 mm to about 3 cm, preferably about 15 mm.

Modifications and variations of the methods and devices described herein will be obvious to those skilled in the art from the foregoing detailed description. Such modifications and variations are intended to come within the scope of the appended claims.

1. A device for applying microneedles to skin or other biological tissue, the device comprising:
an applicator portion which comprises (i) a support structure from which an array of microneedles extend, and (ii) a release mechanism; and
a base portion configured to rest against the skin or other biological tissue and hold the applicator portion in a pre-launch position in which the microneedles face the skin or other biological tissue a spaced distance away from a surface of the skin or other biological tissue,
wherein the applicator portion is configured to receive a manually applied force which, upon exceeding a predetermined threshold, causes the release mechanism to trigger and release the microneedles together with at least part of the support structure from the pre-launch position, thereby driving the microneedles along a guide path toward the skin or other biological tissue at a force and a velocity effective to insert the microneedles into the skin or other biological tissue.

2. The device of claim 1, wherein the base portion comprises a stabilization portion configured to be placed against a patient's skin such that the guide path is perpendicular to a surface of the skin at a target site of insertion of the microneedles.

3. The device of claim 1, wherein the base portion comprises one or more outer walls defining an opening in which the microneedles together with at least part of the support structure are configured to travel along the guide path.

4. The device of claim 3, wherein the one or more outer walls comprises one or more guiderails or slots that matingly engage with one or more slots or guiderails of the applicator portion, respectively.

5. The device of claim 3, wherein the opening defined by the one or more walls of the base portion is cylindrical and the applicator portion comprises an elongated cylindrical body configured to translate within the opening.

6. The device of claim 1, wherein the release mechanism is configured to trigger by mechanical fracture of a portion of the support structure.

7. The device of claim 6, wherein the support structure comprises a polymeric film.

8. The device of claim 6, wherein the release mechanism comprises a plurality of perforations or predefined lines of weakness in the support structure.

9. The device of claim 1, wherein the release mechanism is configured to trigger by deformation of a latch feature.

10. The device of claim 9, wherein the latch feature comprises a lip on the applicator portion that, in the pre-launch position, interferes with a ledge on a wall of the base portion.

11. The device of claim 1, wherein the release mechanism is integral with the support structure.

12. The device of claim 1, wherein the release mechanism is separate from the support structure.

13. The device of claim 12, wherein the applicator portion comprises an elongated body having an upper end portion configured to receive a manually applied force and a lower end portion comprising the array of microneedles, where the release mechanism is disposed between the upper end portion and the lower end portion.

14. The device of claim 1, which is configured to produce a velocity effective to insert the microneedles that is from 1 m/s to 15 m/s.

15. The device of claim 1, wherein the predetermined threshold of the manually applied force is from N.

16. The device of claim 1, wherein the device is configured to apply more than one array of microneedles.

17-18. (canceled)

19. A system comprising:
an insertion tool comprising:
a housing; and
a backing releasably attached to the housing; and
an array of microneedles comprising a substance of interest, wherein the array of microneedles is provided on a bottom side of the backing;
wherein a user-applied force to the backing is effective to separate the backing from the housing and to insert the array of microneedles to a biological tissue at a predetermined velocity.

20-30. (canceled)

31. A method comprising:
positioning a base of an applicator tool against a target tissue surface wherein an array of microneedles extend from an applicator disposed in a pre-launch position within the base, with the microneedles facing the target tissue surface a spaced distance away therefrom by at least 5 mm;
manually applying a force to an upper surface of the applicator tool to exceed a predetermined threshold, causing a release mechanism connecting the applicator to the base to trigger and release the microneedles together with at least part of the applicator, thereby driving, via the manually applied force, the microneedles along a guide path toward the tissue surface at a force and a velocity effective to insert the microneedles into the skin or other biological tissue,
wherein the triggering of the release mechanism comprises (i) mechanical fracture of a structure securing the applicator in the pre-launch position, or (ii) deformation of a latch feature.

32-35. (canceled)

36. A device for applying microneedles to skin or other biological tissue, the device comprising:
an applicator which comprises an array of microneedles; and
a trigger mechanism operably connected to the applicator,
wherein the device is configured to apply the array of microneedles to skin with a controlled velocity, force, and angle, and
wherein the device has no stored energy and the trigger mechanism does not permit the array of microneedles to be displaced toward to the skin until a threshold manual force applied to the device.

37. The device of claim 36, further comprising a base connected to the applicator and trigger mechanism and configured to be placed against a skin surface and stabilize a position of the applicator relative to the skin until the threshold manual force is applied to the device, wherein, in the position, the microneedles are spaced a distance away from the skin from about 5 mm to about 3 cm.

38-39. (canceled)
